SEQUENCE LISTING (19 sequences)

SEQ ID NO: 1 - protein, 32 aa, Paenibacillus kyungheensis
  2,3-didehydroalanine (5)..(5)
  alpha-aminobutyric acid (8)..(8)
  alpha-aminobutyric acid (13)..(13)
  (Z)-2,3-didehydobutyrine (18)..(18)
  alpha-aminobutyric acid (23)..(23)
  alpha-aminobutyric acid (25)..(25)
  2,3-didehydroalanine (31)..(31)
WKAQXFAXPGAVXGVLQXAFIQXAXANAHIXK

SEQ ID NO: 2 - protein, 32 aa, Lactococcus lactis
  2,3-didehydroalanine (5)..(5)
  alpha-aminobutyric acid (8)..(8)
  alpha-aminobutyric acid (13)..(13)
  (Z)-2,3-didehydobutyrine (18)..(18)
  alpha-aminobutyric acid (23)..(23)
  alpha-aminobutyric acid (25)..(25)
  2,3-didehydroalanine (31)..(31)
WKAEXLAXPGAVXGALQXAFLQXLXANAKIXK

SEQ ID NO: 3 - protein, 34 aa, Bacillus subtilis
  (Z)-2,3-didehydobutyrine (2)..(2)
  2,3-didehydroalanine (5)..(5)
  alpha-aminobutyric acid (8)..(8)
  alpha-aminobutyric acid (13)..(13)
  alpha-aminobutyric acid (23)..(23)
  alpha-aminobutyric acid (25)..(25)
  2,3-didehydroalanine (33)..(33)
IXAIXLAXPGAKXGALMGANMKXAXAHASIHVXK

SEQ ID NO: 4 - protein, 28 aa, Paenibacillus kyungheensis
  2,3-didehydroalanine (5)..(5)
  alpha-aminobutyric acid (8)..(8)
  alpha-aminobutyric acid (13)..(13)
  (Z)-2,3-didehydobutyrine (18)..(18)
  alpha-aminobutyric acid (23)..(23)
  alpha-aminobutyric acid (25)..(25)
WKAQXFAXPGAVXGVLQXAFIQXAXANA

SEQ ID NO: 5 - protein, 22 aa, Paenibacillus kyungheensis
  2,3-didehydroalanine (5)..(5)
  alpha-aminobutyric acid (8)..(8)
  alpha-aminobutyric acid (13)..(13)
  (Z)-2,3-didehydobutyrine (18)..(18)
WKAQXFAXPGAVXGVLQXAFIQ

SEQ ID NO: 6 - protein, 19 aa, Paenibacillus kyungheensis
  2,3-didehydroalanine (5)..(5)
  alpha-aminobutyric acid (8)..(8)
  alpha-aminobutyric acid (13)..(13)
  (Z)-2,3-didehydobutyrine (18)..(18)
WKAQXFAXPGAVXGVLQXA

SEQ ID NO: 7 - protein, 12 aa, Paenibacillus kyungheensis
  2,3-didehydroalanine (5)..(5)
  alpha-aminobutyric acid (8)..(8)
WKAQXFAXPGAV

SEQ ID NO: 8 - protein, 11 aa, Paenibacillus kyungheensis
  2,3-didehydroalanine (5)..(5)
  alpha-aminobutyric acid (8)..(8)
WKAQXFAXPGA

SEQ ID NO: 9 - protein, 7 aa, Paenibacillus kyungheensis
  2,3-didehydroalanine (5)..(5)
WKAQXFA

SEQ ID NO: 10 - protein, 25 aa, Paenibacillus kyungheensis
  alpha-aminobutyric acid (1)..(1)
  alpha-aminobutyric acid (6)..(6)
  (Z)-2,3-didehydobutyrine (11)..(11)
  alpha-aminobutyric acid (16)..(16)
  alpha-aminobutyric acid (18)..(18)
  2,3-didehydroalanine (24)..(24)
XPGAVXGVLQXAFIQXAXANAHIXK

SEQ ID NO: 11 - protein, 21 aa, Paenibacillus kyungheensis
  alpha-aminobutyric acid (2)..(2)
  (Z)-2,3-didehydobutyrine (7)..(7)
  alpha-aminobutyric acid (12)..(12)
  alpha-aminobutyric acid (14)..(14)
  2,3-didehydroalanine (20)..(20)
VXGVLQXAFIQXAXANAHIXK

SEQ ID NO: 12 - protein, 20 aa, Paenibacillus kyungheensis
  alpha-aminobutyric acid (1)..(1)
  (Z)-2,3-didehydobutyrine (6)..(6)
  alpha-aminobutyric acid (11)..(11)
  alpha-aminobutyric acid (13)..(13)
  2,3-didehydroalanine (19)..(19)
XGVLQXAFIQXAXANAHIXK

SEQ ID NO: 13 - protein, 13 aa, Paenibacillus kyungheensis
  alpha-aminobutyric acid (4)..(4)
  alpha-aminobutyric acid (6)..(6)
  2,3-didehydroalanine (12)..(12)
FIQXAXANAHIXK

SEQ ID NO: 14 - protein, 10 aa, Paenibacillus kyungheensis
  alpha-aminobutyric acid (1)..(1)
  alpha-aminobutyric acid (3)..(3)
  2,3-didehydroalanine (9)..(9)
XAXANAHIXK

SEQ ID NO: 15 - protein, 4 aa, Paenibacillus kyungheensis
  2,3-didehydroalanine (3)..(3)
HIXK

SEQ ID NO: 16 - protein, 21 aa, Paenibacillus kyungheensis
  alpha-aminobutyric acid (1)..(1)
  alpha-aminobutyric acid (6)..(6)
  (Z)-2,3-didehydobutyrine (11)..(11)
  alpha-aminobutyric acid (16)..(16)
  alpha-aminobutyric acid (18)..(18)
XPGAVXGVLQXAFIQXAXANA

SEQ ID NO: 17 - protein, 16 aa, Paenibacillus kyungheensis
  alpha-aminobutyric acid (1)..(1)
  alpha-aminobutyric acid (6)..(6)
  (Z)-2,3-didehydobutyrine (11)..(11)
  alpha-aminobutyric acid (16)..(16)
XPGAVXGVLQXAFIQX

SEQ ID NO: 18 - protein, 15 aa, Paenibacillus kyungheensis
  alpha-aminobutyric acid (1)..(1)
  alpha-aminobutyric acid (6)..(6)
  (Z)-2,3-didehydobutyrine (11)..(11)
XPGAVXGVLQXAFIQ

SEQ ID NO: 19 - protein, 12 aa, Paenibacillus kyungheensis
  alpha-aminobutyric acid (1)..(1)
  alpha-aminobutyric acid (6)..(6)
  (Z)-2,3-didehydobutyrine (11)..(11)
XPGAVXGVLQXA

(19) United States
(12) Patent Application Publication
(10) Pub. No.: US 2022/0133846 A1
(43) Pub. Date: May 5, 2022

(54) NOVEL ANTIMICROBIAL COMPOUND AND USES THEREOF

(71) Applicant: Fraunhofer USA Inc., Newark, DE (US)

(72) Inventors: Jerzy Karczewski, Newtown Square, PA (US); Stephen John Streatfield, Kennett Square, PA (US); Yukari Maezato, Middletown, DE (US); Stephen Peter Krasucki, Newark, DE (US); Christine Mikel Brown, Newark, DE (US); Vidadi Yusibov, Newark, DE (US)

(73) Assignee: Fraunhofer USA Inc., Newark, DE (US)

(21) Appl. No.: 17/434,254

(22) PCT Filed: Mar. 6, 2020
(86) PCT No.: PCT/US2020/021396

§ 371 Date: Aug. 26, 2021

Related U.S. Application Data
(60) Provisional application No. 62/815,644, filed on Mar. 8, 2019.
(60) Provisional application No. 62/868,251, filed on Jun. 28, 2019.

(51) Int. Cl. A61K 38/16; A01N 63/25; A01P 1/00; A61P 31/04; A61K 45/06

(52) U.S. Cl. CPC A61K 38/164; A01N 63/25; A61K 45/06; A61P 31/04; A01P 1/00

(57) ABSTRACT

A novel lantibiotic peptide and a composition comprising the peptide in an antimicrobial effective amount are provided. A method for preparing the composition from a medium into which host cells produce the peptide is also provided. A method of inhibiting growth of microbial cells, killing microbial cells or treating a subject infected by microbial cells, comprising administering to the microbial cells or the subject an effective amount of the peptide or the composition is further provided. Where the microbial cells are on a surface, the method may further comprise administering the peptide or the composition to the surface.

### CROSS-REFERENCE TO RELATED APPLICATION

This application claims priority to United States Provisional Application Nos. 62/815,644, filed Mar. 8, 2019, and 62/868,251, filed Jun. 28, 2019, the contents of all of which are incorporated herein by reference in their entireties for all purposes.

The Sequence Listing for this application is labeled “FCMB-112WO_SequenceListing.txt” which was created on Mar. 6, 2020 and is 9.30 KB. The entire content of the sequence listing is incorporated herein by reference in its entirety.

### FIELD OF THE INVENTION

This invention relates to identification of a novel antimicrobial peptide and use of the antimicrobial peptide to inhibit growth of microbial cells.

### BACKGROUND OF THE INVENTION

Despite ongoing efforts, the number of new antibiotics approved annually in the United States continues to decline. In addition, few new antibiotics are in late-phase clinical trials, and nearly all belong to existing classes. On the other hand, infections caused by multi-drug resistant (MDR) pathogens are continually on the rise. The most recent relevant examples are found among bacteria described by the acronym ESKAPE pathogens (Enterococcus faecium, Staphylococcus aureus, Klebsiella pneumoniae, Acinetobacter baumannii, Pseudomonas aeruginosa, and Enterobacter species).

Over 90% of wounds contain biofilms, which include well-organized bacterial communities embedded in an extracellular polymeric matrix. Biofilms may become resistant to therapeutic treatment shortly after their development. Therefore, antibacterial agents active against biofilms could prevent or greatly reduce bacterial infections.

Today, natural products continue to play an important role in the drug discovery process, due to their structural diversity and complexity. Antimicrobial peptides (AMPs) are a growing class of natural and synthetic oligopeptides and present a promising area for the discovery of new antibiotics. AMPs have been shown to be effective against a wide spectrum of targets including viruses, bacteria, fungi, and parasites. AMPs typically have a net positive charge, allowing them to selectively interact with anionic bacterial membranes and with other negatively charged cell structures, which leads to membrane disruption and/or protein, DNA or RNA synthesis inhibition. AMPs are generally effective against either bacteria or fungi but can have different modes of action against different types of pathogens. Natural AMPs are produced by prokaryotes (e.g., bacteria) and eukaryotes (e.g., plants, fungi, and animals). More than 5,000 AMPs had been discovered or synthesized as of 2013. Nisin and subtilin are the most prominent AMPs and show an antimicrobial activity in the nanomolar range against a broad spectrum of Gram-positive bacteria. Nisin, also known as E 234, is a food additive that carries the Generally Regarded as Safe (GRAS) designation.

Several lantibiotics, i.e., peptide antibiotics that contain the characteristic amino acid lanthionine or methyllanthionine, have demonstrated excellent in vivo activities and are being evaluated for further development. Efficacy equal to vancomycin was demonstrated for the semisynthetic lantibiotic NVB333 against a Methicillin-resistant Staphylococcus aureus (MRSA) strain in a bronchoalveolar infection model. Furthermore, a recent study indicated that lantibiotics are effective for treatment of S. aureus-induced skin infections and can accelerate wound closure. However, several characteristics of lantibiotics, such as instability and/or insolubility at physiological pH, and susceptibility to proteolytic digestion and other chemical modifications leading to attenuated activity, have limited their further development and/or evaluation in the clinic. Nisin has been used in the food industry for many years and has proven safe. In food applications, nisin-producing bacteria are incorporated into the process as adjunct cultures and therefore the product does not require extensive processing. Nisin production in L. lactis can reach 100 mg/L and be further optimized. However, solubility of nisin at physiological pH decreases drastically, complicating its purification and formulation for pharmaceutical applications. In addition, oxidation or succinylation observed in some lantibiotics (e.g., nisin and subtilin) leads to loss of activity further confounding development of these otherwise promising molecules. To remediate this, attempts to generate more stable lantibiotics by site-directed mutagenesis have been undertaken and have demonstrated that even minor changes in secondary structure, such as altering the K12 residue of nisin A, can generate derivatives with a markedly enhanced antimicrobial activity.

There remains a need for a novel antimicrobial peptide effective against a wide range of bacteria, especially those in biofilms.

### SUMMARY OF THE INVENTION

The present invention relates to a novel lantibiotic, CMB001, and its uses and preparation. The inventors have surprisingly discovered that, unlike other lantibiotics such as subtilin and nisin, CMB001 retains an antimicrobial activity under physiological conditions, for example, at a pH around 7 or higher, and/or in the presence of plasma, serum or whole blood and it is active against biofilms. CMB001 shows in vivo efficacy in a murine model of infection by antibiotic-resistant bacteria.

A method of inhibiting growth of microbial cells is provided. The inhibition method comprises administering to the microbial cells an effective amount of a composition comprising a peptide. The peptide consists of an amino acid sequence selected from the group consisting of SEQ ID NOs: 1 and 4-19 or an amino acid sequence at least 90% homologous to the amino acid sequence selected from the group consisting of SEQ ID NOs: 1 and 4-19. In one embodiment, the peptide consists of SEQ ID NO: 1.

A method of killing microbial cells is provided. The killing method comprises administering to the microbial cells an effective amount of a composition comprising a peptide. The peptide consists of an amino acid sequence selected from the group consisting of SEQ ID NOs: 1 and 4-19 or an amino acid sequence at least 90% homologous to the amino acid sequence selected from the group consisting of SEQ ID NOs: 1 and 4-19. In one embodiment, the peptide consists of SEQ ID NO: 1.

A method of treating a subject infected by microbial cells is provided. The treatment method comprises administering to the subject an effective amount of a composition comprising a peptide. The peptide consists of an amino acid sequence selected from the group consisting of SEQ ID NOs: 1 and 4-19 or an amino acid sequence at least 90% homologous to an amino acid sequence selected from the group consisting of SEQ ID NOs: 1 and 4-19. In one embodiment, the peptide consists of SEQ ID NO: 1.

For each of the inhibition, killing or treatment method of the present invention, the microbial cells may be selected from the group consisting of Staphylococcaceae, Streptococcaceae, Enterococcaceae, Moraxellaceae, Peptostreptococcaceae, Mycobacteriaceae, Pseudomonadaceae, Enterobacteriaceae, Bacillaceae, Yersiniaceae, fungi and combinations thereof. The microbial cells may be selected from the group consisting of Staphylococcus, Streptococcus, Enterococcus, Acinetobacter, Clostridioides, Mycobacterium, Escherichia, Pseudomonas, Klebsiella, Bacillus and Yersinia. The microbial cells may be of a single-drug resistant strain. The single drug resistant strain may be methicillin-resistant Staphylococcus aureus (MRSA). The microbial cells may be of a multi-drug resistant strain. The multi-drug resistant strain may be an S. aureus strain.

For each of the inhibition, killing or treatment method, the composition may further comprise an additional antimicrobial agent. The additional antimicrobial agent may be selected from the group consisting of cephalosporins, carbapenems, macrolides, aminoglycosides, quinolones, sulfonamides, tetracyclines and combinations thereof. The composition may further comprise a potentiator. The potentiator may be selected from the group consisting of polymyxin-derived peptides, β-lactamase inhibitors and combinations thereof. The composition may further comprise a stabilizer. The stabilizer may be selected from the group consisting of a salt, a chelating agent, a polypeptide, a lipid and a nanoparticle. The chelating agent may be EDTA or EGTA.

For the treatment method, the subject may be a mammal. The mammal may be a human.

Where the microbial cells are in a biofilm, the inhibition or killing method may further comprise administering the composition into the biofilm.

Where the microbial cells are on a surface, the inhibition or killing method may further comprise administering the composition to the surface. The surface may be on a medical device or medical equipment. The medical device may be an implant or catheter.

An isolated peptide is also provided. The isolated peptide consists of an amino acid sequence selected from the group consisting of SEQ ID NOs: 1 and 4-19 or an amino acid sequence at least 90% homologous to an amino acid sequence selected from the group consisting of SEQ ID NOs: 1 and 4-19. In one embodiment, the peptide consists of SEQ ID NO: 1.

For each isolated peptide of the present invention, a composition is provided. The composition comprises the peptide in an antimicrobial effective amount. The peptide may be in an amount effective for inhibiting growth of microbial cells. The peptide may be in an amount effective for killing at least 80% of microbial cells. The microbial cells may be selected from the group consisting of Staphylococcaceae, Streptococcaceae, Enterococcaceae, Moraxellaceae, Peptostreptococcaceae, Mycobacteriaceae, Pseudomonadaceae, Enterobacteriaceae, Bacillaceae, Yersiniaceae, fungi and combinations thereof. The microbial cells may be selected from the group consisting of Staphylococcus, Streptococcus, Enterococcus, Acinetobacter, Clostridioides, Mycobacterium, Escherichia, Pseudomonas, Klebsiella, Bacillus and Yersinia. The microbial cells may be of a single-drug resistant strain. The single drug resistant strain may be methicillin-resistant Staphylococcus aureus (MRSA). The microbial cells may be of a multi-drug resistant strain. The multi-drug resistant strain may be an S. aureus strain.

The composition may further comprise an additional antimicrobial agent. The additional antimicrobial agent may be selected from the group consisting of cephalosporins, carbapenems, macrolides, aminoglycosides, quinolones, sulfonamides, tetracyclines and combinations thereof. The composition may further a potentiator. The potentiator may be selected from the group consisting of polymyxin-derived peptides, β-lactamase inhibitors and combinations thereof. The composition may further comprise a stabilizer. The stabilizer may be selected from the group consisting of a salt, a chelating agent, a polypeptide, a lipid and a nanoparticle. The chelating agent may be EDTA or EGTA.

For each composition of the present invention, the microbial cells may be in or on a subject in need thereof. The subject may be a mammal. The microbial cells may be in a biofilm. The microbial cells may be on a surface. The surface may be on a medical device or medical equipment. The medical device may be an implant or catheter.

For each peptide of the present invention, a method for preparing a composition comprising the peptide is provided. The composition is prepared from a medium into which host cells produce the peptide. The preparation method comprises (a) removing host cells from the medium, whereby a clarified medium comprising the peptide is obtained; (b) adsorbing the peptide in the clarified medium onto first resins and desorbing, whereby a first peptide fraction is obtained; (c) adsorbing the peptide in the first peptide fraction onto second resins and desorbing, whereby a second peptide fraction is obtained; and (d) subjecting the second peptide fraction to reversed phase chromatography, whereby a composition comprising the peptide in an antimicrobial effective amount is obtained. The preparation method may further comprise culturing the host cells in the medium until an antibacterial activity is detected in the medium before step (a). The concentration of the peptide in the composition may be at least 100 times greater than that in the medium. The host cells may be selected from the group consisting of Paenibacillaceae, Streptococcaceae, Enterobacteriaceae, Bacillaceae, Saccharomycetaceae and combinations thereof. The host cells may express one or more heterologous enzymes selected from the group consisting of dehydratases, cyclases, proteases, transporters, and combinations thereof.

### BRIEF DESCRIPTION OF THE DRAWINGS

FIG. 1 shows characterization of CMB001: (A) Scanning electron microscope (SEM) image of Paenibacillus kyungheensis producer cells of CMB001; (B) Purified CMB001 analyzed by 4-12% SDS-PAGE stained with Coomassie Blue; (C) Reverse-phase chromatography of purified CMB001; and (D) Mass spectrum of purified CMB001.

FIG. 2 shows a summary of inter-residue Nuclear Overhauser Effects (NOEs) and prediction of secondary structure of CMB001 using 1Hα chemical shifts. The sequence of CMB001 is depicted on top of the graphics. dA stands for 2,3-didehydroalanine, dB stands for (Z)-2,3-didehydobutyrine and Ab stands for α-aminobutyric acid. Legend labels are as follows: dαN indicates residues with Hα (i) to HN (i+1) NOE connections, dNN indicates residues with HN (i) to HN (i+1) NOE connections, dβN indicates residues with Hβ (i) to HN (i+1) NOE connections, dαN (i,i+3) indicates residues with Hα (i) to HN (i+3) NOE connections, dap (i,i+3) indicates residues with Hα (i) to Hβ (i+3) NOE connections, dαN (i,i+4) indicates residues with Hα (i) to HN (i+4) NOE connections, dNN indicates residues with HN (i) to HN (i+2) NOE connections, dαN (i,i+2) indicates residues with Hα (i) to HN (i+2) NOE connections, Δδ(1Hα) indicates 1Hα difference in chemical shift to sequence adjusted random coil chemical shift for residue type, Chemical Shift Index (CSI) indicates CSI values for residues where −1 indicates α-helix and +1 indicates β-sheet. For the (i,i+1) NOE connections, the thickness of the bar indicates the intensity of the NOE cross peak. At the bottom is shown the secondary structure for the peptide.

FIG. 3 shows the amino acid sequences of CMB001 and its biosynthetic analogs CMB0011-0027. The amino acid sequence of CMB001 (SEQ ID NO: 1) is compared to that of the two most studied lantibiotics: subtilin (SEQ ID NO:2) (A) and nisin (SEQ ID NO: 3) (B), as well as biosynthetic analogs with anticipated antimicrobial activity (C): CMB001-1 (SEQ ID NO: 4), CMB001-2 (SEQ ID NO: 5), CMB001-3 (SEQ ID NO: 6), CMB001-4 (SEQ ID NO: 7), CMB001-5 (SEQ ID NO: 8), CMB001-6 (SEQ ID NO: 9), CMB001-7 (SEQ ID NO: 10), CMB001-8 (SEQ ID NO: 11), CMB001-9 (SEQ ID NO: 12), CMB001-10 (SEQ ID NO: 13), CMB001-11 (SEQ ID NO: 14), CMB001-12 (SEQ ID NO: 15), CMB001-13 (SEQ ID NO: 16), CMB001-14 (SEQ ID NO: 17), CMB001-15 (SEQ ID NO: 18), and CMB001-16 (SEQ ID NO: 19). Amino acids in CMB001 that differ from those in subtilin (A) or nisin (B) are bolded. dA stands for 2,3-didehydroalanine, dB stands for (Z)-2,3-didehydobutyrine and Ab stands for α-aminobutyric acid.

FIG. 4 shows cartoon representation of the 3D structure ensemble of CMB001. (A) Overlay of 8 structures out of 15 chosen as a representative ensemble based on distance restraint violations and a converged backbone Root-Mean-Square Deviation (RMSD). (B) A Single structure ensemble with the N-terminal Trp-1 and C-terminal Lys-32 residues labelled.

FIG. 5 shows the effect of CMB001 treatment on S. aureus and MRSA biofilms. (A) Effect of CMB001 and vancomycin on viability of pre-formed S. aureus (SA) and MRSA biofilms. (B) Effect of pre-coating with CMB001 for 1 or 24 hours on S. aureus viability.

FIG. 6 shows time-kill curves to determine bactericidal or bacteriostatic activity of CMB001 against (A) drug-susceptible S. aureus (SA) and MRSA, and (B) drug-susceptible CH-40 A. baumannii and drug-resistant CH46 A. baumannii.

FIG. 7 shows scanning electron microscope (SEM) images of S. aureus (top panels) and A. baumannii (bottom panels) untreated (C) (left panels) or treated for 10 minutes (10 min) (middle panels) or 60 minutes (60 min) (right panels) with CMB001 at 4× Minimum Inhibitory Activity (MIC). The arrows indicate bleb-like structures (top right panel) and undulating deformations and folds (bottom middle and right panels).

FIG. 8 shows scanning electron microscope (SEM) images of M. smegmatis untreated (PBS) or treated with CMB001 at 1×MIC, 2×MIC or 4×MIC for 60 minutes.

FIG. 9 shows cytotoxicity of CMB001 and ciprofloxacin to J774A mouse BALB/c cells.

FIG. 10 shows solubility of 1 mg/mL solutions of CMB001 and nisin after 10-minute exposures to pH values ranging from 3-9.

FIG. 11 shows in vivo efficacy of CMB001 against methicillin-resistant S. aureus (MRSA) in a murine thigh wound model of infection. An increase in S. aureus in the thigh from 3.3 log 10 cfu/g to 7.28×10⁷ cfu/g (A) or from 4.1 log 10 cfu/g to 3.5×10⁸ cfu/g (B) was achieved in vehicle treated animals. In two separate experiments CMB001 treatment groups were administered three times a day intravenously over dose ranges of 0.5-10 mg/kg (A) or 5-30 mg/kg (B). Treatment with CMB001 led to a dose-dependent reduction of S. aureus in the thigh when compared to vehicle treated mice. Treatment with 25 mg/kg vancomycin provided a comparator positive control.

### DETAILED DESCRIPTION OF THE INVENTION

The present invention provides a novel lantibiotic, CMB001, and its uses and preparation. CMB001 is a polycyclic peptide antibiotic containing (methyl)lanthionines, which may be introduced post-translationally into a prepropeptide by biosynthetic enzymes. The invention is based on the unexpected discovery of an isolated novel peptide having a stable antimicrobial activity against various microbial cells and biofilms and showing no or low toxicity to mammalian cells. Unlike many well studied lantibiotics, the novel antimicrobial peptide CMB001 is stable under physiological conditions, for example, at a pH around 7 or higher, and/or in the presence of plasma, serum or whole blood.

The terms “isolated” and “purified” are used herein interchangeably, and refer to an agent, for example, a biological molecule, a chemical compound or a combination thereof, that is separated, isolated or purified from an environment in which the agent exists naturally. In other words, the isolated or purified molecule or compound does not exist in a natural environment.

The term “antimicrobial” or “antimicrobial activity” used herein refers to a biological activity of an agent, for example, a biological molecule, a chemical compound or a combination thereof, that prevents or inhibits (or reduces) the growth of, or kills cells of one or more microorganisms, also called microbial cells. Examples of microorganism include Gram-positive and/or Gram negative bacteria strains, especially those related to currently known antibiotic resistant strains. The term “antibiotic” used herein refers to an agent, for example, a biological molecule, a chemical compound or a combination thereof, having an antimicrobial activity.

The term “peptide” used herein refers to a polymer having 4-50 amino acid residues. The term “lantibiotic peptide” used herein refers to a peptide having an antimicrobial activity and comprising one or more amino acids such as lanthionine and methyllanthionine.

The term “potentiator” used herein refers to an agent, for example, a biological molecule, a chemical compound or a combination thereof, that increases a biological activity of another agent. The increase may be at least 10%, 20%, 30%, 40%, 50%, 60%, 70%, 80%, 90%, 100% or 200%. The biological activity may be an antimicrobial activity against a microbe. The potentiator may or may not have an antimicrobial activity, which antimicrobial activity may be weak.

An isolated peptide is provided. The isolated peptide consists of an amino acid sequence selected from the group consisting of SEQ ID NOs: 1 and 4-19 or an amino acid sequence at least 70%, 75%, 80%, 85%, 90%, 95%, 99% or 100% homologous to an amino acid sequence selected from the group consisting of SEQ ID NOs: 1 and 4-19. In one embodiment, the isolated peptide is WKAQX₁FAX₃PGAVX₃GVLQX₂AFIQX₃AX₃ANAHIX₁K (SEQ ID NO: 1), wherein X₁ is 2,3-didehydroalanine, X₂ is (Z)-2,3-didehydobutyrine and X₃ is α-aminobutyric acid. In another embodiment, the isolated peptide is a fragment of WKAQX₁FAX₃PGAVX₃GVLQX₂AFIQX₃AX₃ANAHIX₁K (SEQ ID NO: 1), wherein X₁ is 2,3-didehydroalanine, X₂ is (Z)-2,3-didehydobutyrine and X₃ is α-aminobutyric acid, for example, consisting of any one of SEQ ID NO: 4-19. The isolated peptide may be antimicrobial.

A composition comprising an antimicrobial effective amount of the isolated peptide of the present invention is provided.

The isolated peptide in the composition is stable. The term “stable” or “stability” used herein refers to a small loss (e.g., less than 30%, 20%, 10%, 5% or 1%) of the isolated peptide in the composition or its biological activity (e.g., antimicrobial activity) under predetermined conditions (e.g., pH or temperature) after a predetermined period of time. The predetermined conditions may include a pH of 3-9, 4-9, 5-9, 6-9, 7-9, 8-9, 3-8, 4-8, 5-8, 6-8, 7-8, 3-7, 4-7, 5-7, 6-7, 3-6, 4-6, or 5-6, or greater than 6 or 7. The predetermined conditions may include a temperature of 4-60, 4-50, 4-40, 4-30, 4-25, 4-20, 4-15 or 4-10° C. The predetermined period of time may be 1, 2, 3, 4, 5, 6 or 7 days, or 2, 4, 6 or 8 weeks.

At least 70%, 80%, 90%, 95%, 99% or 100% of the isolated peptide may remain in the composition at a predetermined pH (e.g., 3-9, 4-9, 5-9, 6-9, 7-9, 8-9, 3-8, 4-8, 5-8, 6-8, 7-8, 3-7, 4-7, 5-7, 6-7, 3-6, 4-6, or 5-6, or greater than 6 or 7) after a predetermined period of time (e.g., 1, 2, 3, 4, 5, 6 or 7 days, or 2, 4, 6 or 8 weeks). In one embodiment, at least 70%, 80%, 90%, 95%, 99% or 100% of the antimicrobial activity of the isolated peptide in the composition may remain at a pH greater than 7 after a predetermined period of time (e.g., 1, 2, 3, 4, 5, 6 or 7 days, or 2, 4, 6 or 8 weeks).

At least 70%, 80%, 90%, 95%, 99% or 100% of the isolated peptide may remain in the composition at a predetermined temperature (e.g., 4-60, 4-50, 4-40, 4-30, 4-25, 4-20, 4-15 or 4-10° C.) after a predetermined period of time (e.g., 1, 2, 3, 4, 5, 6 or 7 days, or 2, 4, 6 or 8 weeks). In one embodiment, at least 70%, 80%, 90%, 95%, 99% or 100% of the antimicrobial activity of the isolated peptide in the composition may remain at a temperature of 4-60° C. after a predetermined period of time (e.g., 1, 2, 3, 4, 5, 6 or 7 days, or 2, 4, 6 or 8 weeks).

In the composition, the peptide may be in an amount effective for inhibiting growth of microbial cells. The growth of the microbial cells may be inhibited by at least 10%, 20%, 30%, 40%, 50%, 60%, 70%, 80%, 90%, 95%, 99% or 100%. The growth of the microbial cells may be inhibited for a predetermined period of time (e.g., 1, 2, 3, 4, 5, 6 or 7 days, or 2, 4, 6 or 8 weeks).

In the composition, the peptide may be in an amount effective for killing microbial cells. At least 10%, 20%, 30%, 40%, 50%, 60%, 70%, 80%, 90%, 95%, 99% or 100% of the microbial cells may be killed. The microbial cells may be killed within a predetermined period of time (e.g., 1, 2, 3, 4, 5, 6 or 7 days, or 2, 4, 6 or 8 weeks).

In the composition, the peptide may be in an amount effective for treating a subject infected by microbial cells. At least 10%, 20%, 30%, 40%, 50%, 60%, 70%, 80%, 90%, 95%, 99% or 100% of the microbial cells in a sample from the subject may be killed. The microbial cells may be killed within a predetermined period of time (e.g., 1, 2, 3, 4, 5, 6 or 7 days, or 2, 4, 6 or 8 weeks).

For each composition, the microbial cells may be selected from the group consisting of Staphylococcaceae, Streptococcaceae, Enterococcaceae, Moraxellaceae, Peptostreptococcaceae, Mycobacteriaceae, Pseudomonadaceae, Enterobacteriaceae, Bacillaceae, Yersiniaceae, fungi and combinations thereof. The microbial cells may be selected from the group consisting of Staphylococcus, Streptococcus, Enterococcus, Acinetobacter, Clostridioides, Mycobacterium, Escherichia, Pseudomonas, Klebsiella, Bacillus and Yersinia. The Staphylococcaceae may be Staphylococcus aureus. The Streptococcaceae may be Streptococcus pneumonia. The Enterococcaceae may be Enterococcus faecalis or Enterococcus faecium. The Moraxellaceae may be A. baumannii. The Peptostreptococcaceae may be Clostridioides difficile or Clostridium difficile. The Mycobacteriaceae may be Mycobacterium tuberculosis. The Pseudomonadaceae may be Pseudomonas aeruginosa. The Enterobacteriaceae may be Klebsiella pneumonia. The Bacillaceae may be Bacillus anthracis. The Yersiniaceae may be Yersinia pestis. The fungi may be Fusarium solani. The microbial cells may be of a single-drug resistant strain. The single drug resistant strain may be methicillin-resistant Staphylococcus aureus (MRSA). The microbial cells may be of a multi-drug resistant strain. The multi-drug resistant strain may be a S. aureus strain.

For each composition, the microbial cells may be at any location. For example, the microbial cells may be in or on a subject in need of the composition of the present invention. The subject may be a mammal, for example, a human. The microbial cells may be in a biofilm. The microbial cells may be on a surface. The surface may be on a medical device or medical equipment. The medical device may be an implant or catheter.

The composition may further comprise an additional antimicrobial agent. The additional antimicrobial agent may be selected from the group consisting of cephalosporins, carbapenems, macrolides, aminoglycosides, quinolones, sulfonamides, tetracyclines and combinations thereof.

The composition may further comprise a potentiator. The potentiator may increase the inhibitory, killing or treatment effect of the composition by at least 10%, 20%, 30%, 40%, 50%, 60%, 70%, 80%, 90%, 100% or 200%. The potentiator may be selected from the group consisting of polymyxin-derived peptides, β-lactamase inhibitors and combinations thereof.

The composition of the present invention may further comprise a stabilizer. The stabilizer may increase the stability of the peptide by at least 10%, 20%, 30%, 40%, 50%, 60%, 70%, 80%, 90%, 100% or 200%. The stabilizer may be a salt, a chelating agent, a polypeptide, a lipid, platelet-poor or -rich plasma, serum or a nanoparticle. The chelating agent may be EDTA or EGTA.

A method of inhibiting growth of microbial cells is provided. This inhibition method comprises administering to the microbial cells an effective amount of a composition comprising the isolated peptide of the present invention. The growth of the microbial cells may be inhibited by at least 10%, 20%, 30%, 40%, 50%, 60%, 70%, 80%, 90%, 95%, 99% or 100%. The growth of the microbial cells may be inhibited for a predetermined period of time (e.g., 1, 2, 3, 4, 5, 6 or 7 days, or 2, 4, 6 or 8 weeks). The composition may have a pH of 3-9, 4-9, 5-9, 6-9, 7-9, 8-9, 3-8, 4-8, 5-8, 6-8, 7-8, 3-7, 4-7, 5-7, 6-7, 3-6, 4-6, or 5-6, or greater than 6 or 7. In one embodiment, the composition has a pH greater than 7.

A method of killing microbial cells is provided. This killing method comprises administering to the microbial cells an effective amount of a composition comprising the isolated peptide of the present invention. At least 10%, 20%, 30%, 40%, 50%, 60%, 70%, 80%, 90%, 95%, 99% or 100% of the microbial cells may be killed. The microbial cells may be killed within a predetermined period of time (e.g., 1, 2, 3, 4, 5, 6 or 7 days, or 2, 4, 6 or 8 weeks). The composition may have a pH of 3-9, 4-9, 5-9, 6-9, 7-9, 8-9, 3-8, 4-8, 5-8, 6-8, 7-8, 3-7, 4-7, 5-7, 6-7, 3-6, 4-6, or 5-6, or greater than 6 or 7. In one embodiment, the composition has a pH greater than 7.

A method of treating a subject infected by microbial cells is provided. The treatment method comprises administering to the subject an effective amount of a composition comprising the isolated peptide of the present invention. At least 10%, 20%, 30%, 40%, 50%, 60%, 70%, 80%, 90%, 95%, 99% or 100% of the microbial cells in a sample from the subject may be killed. The microbial cells may be killed within a predetermined period of time (e.g., 1, 2, 3, 4, 5, 6 or 7 days, or 2, 4, 6 or 8 weeks). The composition may have a pH of 3-9, 4-9, 5-9, 6-9, 7-9, 8-9, 3-8, 4-8, 5-8, 6-8, 7-8, 3-7, 4-7, 5-7, 6-7, 3-6, 4-6, or 5-6, or greater than 6 or 7. In one embodiment, the composition has a pH greater than 7.

For each of the inhibition, killing or treatment method of the present invention, the isolated peptide consists of an amino acid sequence selected from the group consisting of SEQ ID NOs: 1 and 4-19 or an amino acid sequence at least 70%, 75%, 80%, 85%, 90%, 95%, 99% or 100% homologous to an amino acid sequence selected from the group consisting of SEQ ID NOs: 1 and 4-19. In one embodiment, the isolated peptide is WKAQX₁FAX₃PGAVX₃GVLQX₂AFIQX₃AX₃ANAHIX₁K (SEQ ID NO: 1), wherein X₁ is 2,3-didehydroalanine, X₂ is (Z)-2,3-didehydobutyrine and X₃ is α-aminobutyric acid. In another embodiment, the isolated peptide is a fragment of WKAQX₁FAX₃PGAVX₃GVLQX₂AFIQX₃AX₃ANAHIX₁K (SEQ ID NO: 1), wherein X₁ is 2,3-didehydroalanine, X₂ is (Z)-2,3-didehydobutyrine and X₃ is α-aminobutyric acid, for example, consisting of an amino acid sequence selected from the group consisting of SEQ ID NOs: 4-19.

For each of the inhibition, killing and treatment method of the present invention, the microbial cells may be selected from the group consisting of Staphylococcaceae, Streptococcaceae, Enterococcaceae, Moraxellaceae, Peptostreptococcaceae, Mycobacteriaceae, Pseudomonadaceae, Enterobacteriaceae, Bacillaceae, Yersiniaceae, fungi and combinations thereof. The microbial cells may be selected from the group consisting of Staphylococcus, Streptococcus, Enterococcus, Acinetobacter, Clostridioides, Mycobacterium, Escherichia, Pseudomonas, Klebsiella, Bacillus and Yersinia. The Staphylococcaceae may be Staphylococcus aureus. The Streptococcaceae may be Streptococcus pneumonia. The Enterococcaceae may be Enterococcus faecalis or Enterococcus faecium. The Moraxellaceae may be A. baumannii. The Peptostreptococcaceae may be Clostridioides difficile or Clostridium difficile. The Mycobacteriaceae may be Mycobacterium tuberculosis. The Pseudomonadaceae may be Pseudomonas aeruginosa. The Enterobacteriaceae may be Klebsiella pneumonia. The Bacillaceae may be Bacillus anthracis. The Yersiniaceae may be Yersinia pestis. The fungi may be Fusarium solani. The microbial cells may be of a single-drug resistant strain. The single drug resistant strain may be methicillin-resistant Staphylococcus aureus (MRSA). The microbial cells may be of a multi-drug resistant strain. The multi-drug resistant strain may be a S. aureus strain.

The inhibition or killing method may further comprise administering to the microbial cells an additional antimicrobial agent. The additional antimicrobial agent may be administered concurrently with, before or after the composition. The peptide and the additional antimicrobial agent may provide a synergistic inhibition effect on the microbial cells. The composition may further comprise the additional antimicrobial agent. The additional antimicrobial agent may be selected from the group consisting of cephalosporins, carbapenems, macrolides, aminoglycosides, quinolones, sulfonamides, tetracyclines and combinations thereof. The inhibition or killing method may further comprise administering to the microbial cells a potentiator. The potentiator may be administered concurrently with, before or after the composition. The potentiator may increase the inhibitory or killing effect of the composition by at least 10%, 20%, 30%, 40%, 50%, 60%, 70%, 80%, 90%, 100% or 200%. The composition may further comprise the potentiator. The potentiator may be selected from the group consisting of polymyxin-derived peptides, β-lactamase inhibitors and combinations thereof.

The inhibition or killing method of the present invention may further comprise administering to the microbial cells a stabilizer. The stabilizer may be administered concurrently with, before or after the composition. The stabilizer may increase the stability of the peptide by at least 10%, 20%, 30%, 40%, 50%, 60%, 70%, 80%, 90%, 100% or 200%. The stabilizer may be a salt, a chelating agent, a polypeptide, a lipid or a nanoparticle. The chelating agent may be EDTA or EGTA.

For each inhibition or killing method of the present invention, the microbial cells may be at any location. Where the microbial cells are in or on a subject in need of the inhibition or killing method, the inhibition or killing method may further comprise administering the composition to the subject. The subject may be a mammal, for example, a human. Where the microbial cells are in a biofilm, the inhibition or killing method may further comprise administering the composition into the biofilm. Where the microbial cells are on a surface, the inhibition or killing method may further comprise administering the composition to the surface. The surface may be on a medical device or medical equipment. The medical device may be an implant or catheter.

The treatment method may further comprise administering to the subject a potentiator. The potentiator may be administered concurrently with, before or after the composition. The potentiator may increase the treatment effect of the composition by at least 10%, 20%, 30%, 40%, 50%, 60%, 70%, 80%, 90%, 100% or 200%. The composition may further comprise the potentiator. The potentiator may be selected from the group consisting of polymyxin-derived peptides, β-lactamase inhibitors and combinations thereof.

The treatment method of the present invention may further comprise administering to the microbial cells a stabilizer. The stabilizer may be administered concurrently with, before or after the composition. The stabilizer may increase the stability of the peptide by at least 10%, 20%, 30%, 40%, 50%, 60%, 70%, 80%, 90%, 100% or 200%. The stabilizer may be a salt, a chelating agent, a polypeptide, a lipid or a nanoparticle. The chelating agent may be EDTA or EGTA.

A method for preparing a composition comprising the isolated peptide of the present invention is provided. The composition is prepared from a medium into which host cells produce the peptide of the present invention. This preparation method comprises removing the host cells from the medium so that a clarified medium (also known as culture supernatant of the host cells) comprising the peptide is obtained; adsorbing the peptide in the clarified medium onto first resins and desorbing so that a first peptide fraction is obtained; adsorbing the peptide in the first peptide fraction onto second resins and desorbing so that a second peptide fraction is obtained; and subjecting the second peptide fraction to reversed phase chromatography such that a composition comprising the peptide is obtained. The composition may comprise the peptide in an antimicrobial effective amount. The host cells may be removed from the medium by centrifugation and/or filtration. The first resins may be hydrophobic resins and the second resins may be ion exchange resins. The first resins may be ion exchange resins and the second resins may be hydrophobic resins. The concentration of the peptide in the composition is at least 5, 10, 20, 30, 40, 50, 60, 70, 80, 90 or 100 times greater than that in the medium. The preparation method may further comprise culturing the host cells in the medium until an antibacterial activity is detected in the medium before removing host cells from the medium. The preparation method may exclude trichloroacetic acid (TCA) precipitation of culture supernatant of the host cells.

According to the preparation method, the host cells may be selected from the group consisting of Paenibacillaceae, Streptococcaceae, Enterobacteriaceae, Bacillaceae, Saccharomycetaceae, and combinations thereof. The Paenibacillaceae may be Paenibacillus. The Streptococcaceae may be Lactococcus. The Enterobacteriaceae may be Escherichia. The Bacillaceae may be Bacilli. The Saccharomycetaceae may be Saccharomyces. The host cells may express one or more heterologous enzymes selected from the group consisting of dehydratases, cyclases, proteases and transporters, and combinations thereof. The dehydratase may be Lan B. The cyclase may be Lna C. The protease may be NisP. The transporter may be NisT. The heterologous enzymes may be derived from organisms of Lactococcus or Paenibacillus genus.

### Example 1. Isolation of CMB001

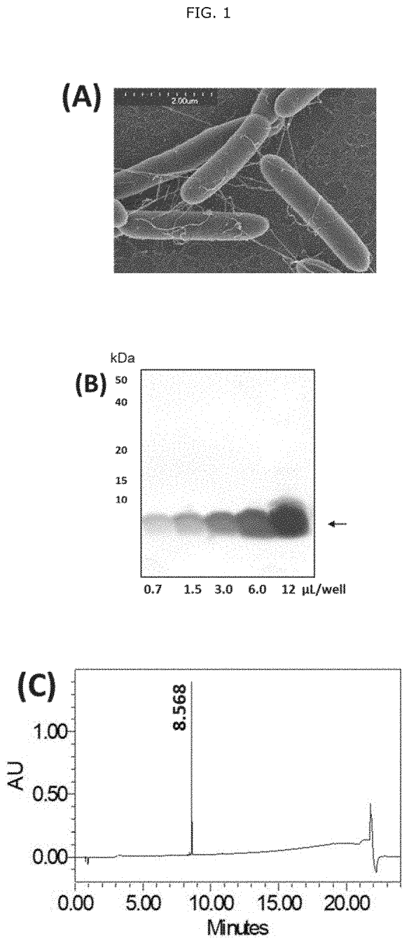

Novel antimicrobial CMB001 was isolated from a culture medium of a bacterial isolate with an antimicrobial activity by purification to homogeneity by a three-step process. The bacterial isolate was identified from a bacterial library by screening for species with an antimicrobial activity. The bacterial isolate (FIG. 1A) is a Gram (+) bacillus having a 100% (16S) homology match with Paenibacillus kyungheensis and may be of the genus Paenibacillus. The cells of the bacterial isolate were removed from the culture medium to generate a clarified medium, which was subsequently subjected to the three-step process. Briefly, the clarified medium was subject to first hydrophobic interaction resin such as Phenyl-Sepharose at pH 6.0, then cation exchange resin such as SP HP at pH 6.0, and lastly reverse phase chromatography using resin such as Source 30RPC, all stages were performed at 22° C. (+/−4° C.).

The isolated CMB001 could be detected by Coomassie Blue when analyzed by SDS-PAGE and its rate of migration indicated a molecular weight of <10 kDa (FIG. 1B), implying CMB001 is a peptide. Analysis of the isolated CMB001 by HPLC revealed a single prominent peak with 95% of area (FIG. 1C). The mass of the isolated CMB001 was 3,346.576 Da as determined by high-resolution mass spectrometry (FIG. 1D). A compound different from CMB001 was previously isolated or purified from the culture medium of the same bacterial isolate when the supernatant of the culture medium was subject to a TCA precipitation in lieu of the three-step process as used to isolate or purify the CMB001 as described above.

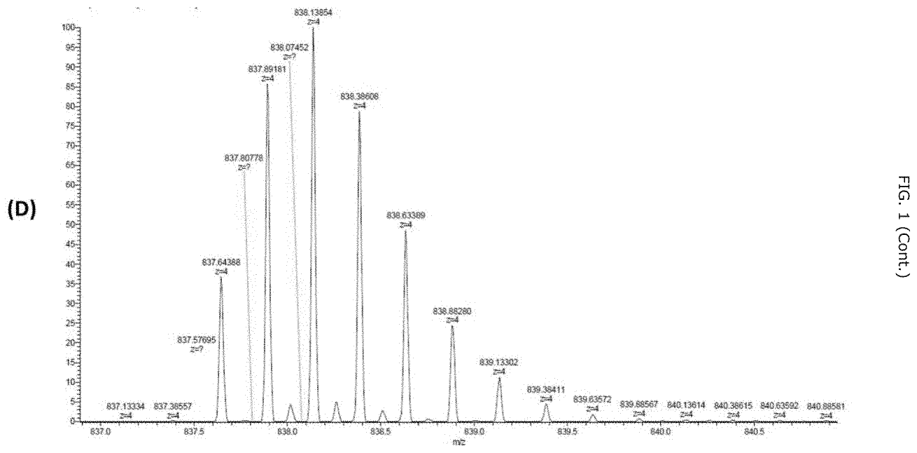

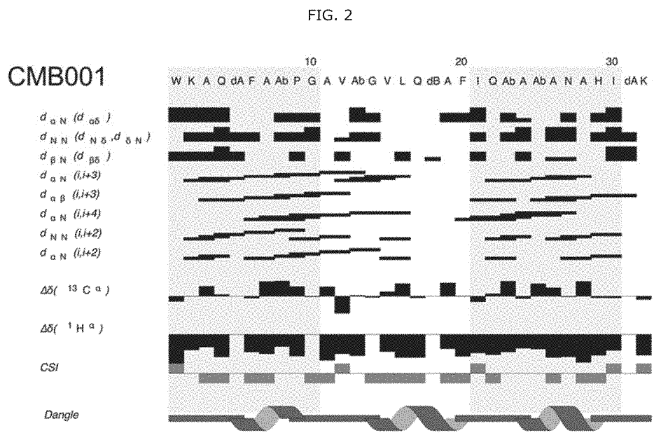

The structure of the isolated CMB001 in DMSO-d6 was elucidated using a combination of 2D experiments with NMR spectroscopy. Using this approach, 98% of all hydrogen chemical shifts could be assigned. The shape and secondary structure content of CMB001 were estimated using a combination of inter-residue NOEs and Hα chemical shifts. CMB001 was predicted as a peptide having three α-helices consisting of residues 3Ala-9Pro, 14Gly-20Phe and 20Gln-29His (FIG. 2).

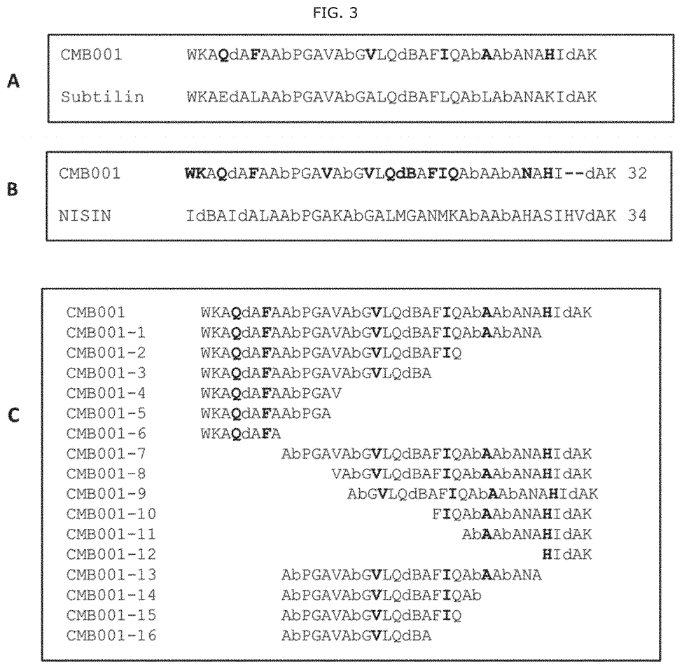

Based on a homology search, and the presence of unnatural amino acids (2,3-didehydroalanine, (Z)-2,3-didehydobutyrine and α-aminobutyric acid) CMB001 could be classified as a novel lantibiotic. CMB001 has an amino acid sequence that has about 81% identity with that of subtilin, including the N-terminal tryptophan (W). The amino acids that differ between CMB001 and subtilin (FIG. 3A) and nisin (FIG. 3B) (bolded) are in the regions proposed to be critical for its antimicrobial activity and define the unique 3D conformation of CMB001. The 3D structure of CMB001 was determined using distance and dihedral angle restraints from assigned NOE cross peaks and chemical shifts. A summary of the structural quality of CMB001 ensemble indicates that the ensemble forms a well-defined 3D-structure consistent with NOE distance restraints and dihedral angle restraints.

Structure calculations were performed, and NOE distance restraints were iterated until a converged structure ensemble was achieved with a backbone RMSD of 0.52 Å. The structure quality is depicted in Table 1.

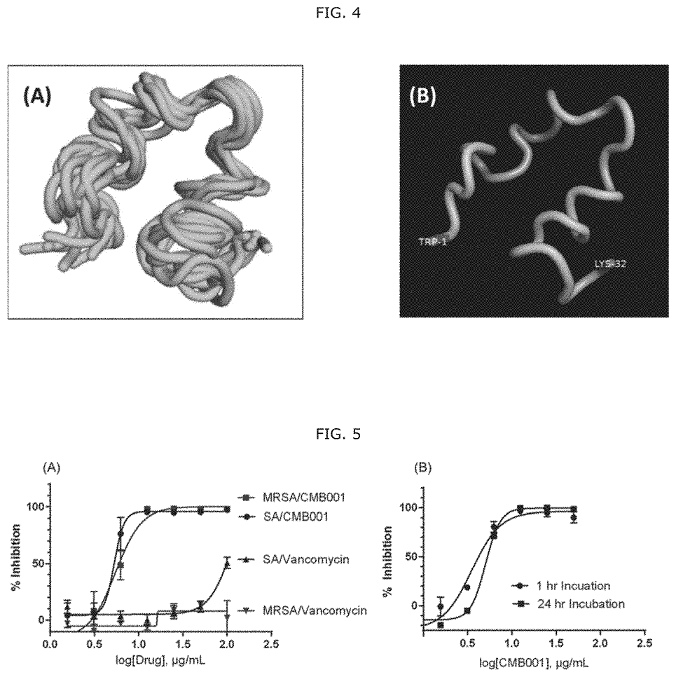

The final 3D structure ensemble forms a U-shaped backbone structure with one α-helix and two pseudo-α-helical regions consisting of residues 14-19 for the α-helix and 3-12 and 20-28 for the N-terminal and C-terminal pseudo-α-helical regions, respectively (FIG. 4). Only one secondary structure element is present in the structure, which is an α-helix consisting of residues 14-19.

A search versus the PDB database revealed a partial match with 1WCO (nisin), but the sequence similarity was limited and thus the structural similarity was also low. The complete structure of subtilin is not available and none of the lantibiotics with known structures derived from solution NMR or X-ray diffraction analysis (1WCO, 2KTN 1MQZ, 2M8V, 1MQX, 1AJ1) show a significant similarity to CMB001 in terms of structure.

### Example 2. Antimicrobial Activity

CMB001 was initially identified as an inhibitor of S. aureus growth. Subsequent antimicrobial profiling was determined using broth micro dilution in 96-well plates, following Clinical & Laboratory Standards Institute guidelines, and revealed that CMB001 inhibited a range of bacteria, including the Gram-positive bacteria S. aureus, E. faecalis and Vancomycin-resistant E. faecium, and the Gram-negative bacteria multi-drug resistant A. baumannii as well as the Mycobacteriaceae Mycobacterium tuberculosis (Table 2).

Peptide content and purity of CMB001 was determined by reversed phase HPLC (Agilent) with detection at 214 nm and compared against CMB001 standard. For standard solution of CMB001, peptide content was determined by quantitative amino acid composition analysis. Minimum Inhibitory Concentration (MIC) is the minimum concentration of an antimicrobial drug required to inhibit visible growth of a microorganism after overnight incubation with the drug. MIC90 is the minimum concentration of an antimicrobial drug required to inhibit 90% of growth of a microorganism after overnight incubation with the drug.

CMB001 was also tested against several panels of clinical isolates, both multidrug resistant (denoted Y) and susceptible (denoted N), obtained from Christiana Hospital, Wilmington, Del., including Staphylococcus (Table 3), Acinetobacter (Table 4) and Enterococcus strains (Table 5). The results including MIC90s are summarized in Table 6.

The anti-biofilm activity of CMB001 was evaluated. Briefly, aliquots of S. aureus (SA) and MRSA in Tryptic Soy Broth were incubated in a 96-well plate for 24 hours, after which the wells were washed to remove planktonic cells. A fresh medium was added, and the plate was further incubated overnight at 32° C. to allow for biofilm formation. CMB001 was then added and incubated with the biofilm for 4 hours. The plate was washed, and cell viability was measured. A dose-dependent reduction in reagent fluorescence was observed, indicating a loss of cell viability with an IC₅₀ of about 4.2 μg/mL (FIG. 5A). In contrast, vancomycin treatment was not effective.

Furthermore, to prevent biofilm formation, wells in a 96-well plate were coated with CMB001, then washed and incubated with S. aureus for 1 or 24 hours. Wells were washed to remove planktonic cells and viability of adherent cells were measured. A dose-dependent reduction in reagent fluorescence was observed with an IC50 of about 1.2 μg/mL (FIG. 5B). By contrast, coating wells with platelet factor 4, a control cationic peptide, had no effect on S. aureus attachment (data not shown).

Taken together, CMB001 is not only an effective antimicrobial agent against MDR planktonic cells, but also a potent anti-biofilm agent capable of killing bacteria upon contact.

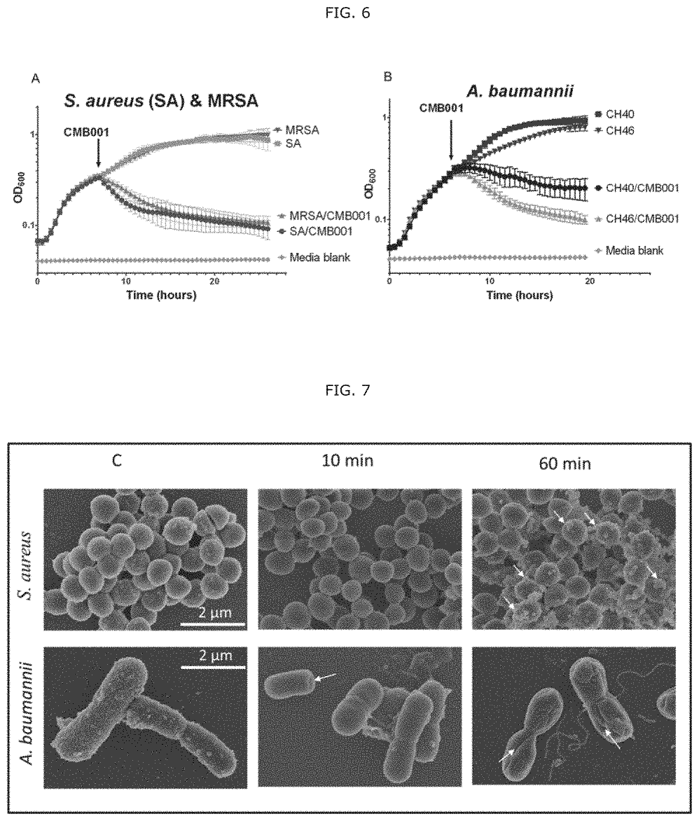

To determine bacteriolytic activity, CMB001 was added at twice the MIC to a test strain of S. aureus or MRSA in mid-exponential growth, and changes in growth were monitored over 20 hours. A marked decrease of OD600 was observed, indicating bacteriolytic activity of CMB001 (FIG. 6A). Similar results were observed for two strains of A. baumannii, CH40 (drug susceptible) and CH-46 (multi-drug resistant) (FIG. 6B).

The frequency of resistance (FoR) was determined in vitro and calculated based on the number of confirmed resistant colonies growing on CMB001-containing media divided by the total number of CFU in the initial test inoculum. At 4×MIC, the frequency of resistance against MRSA and MDR A. baumannii (CH-46) was 4.5×10⁻¹⁰ and 5.2×10⁻⁸, respectively. The frequency of resistance to CMB001 and selected control antibiotics in S. aureus, MRSA and A. baumannii is summarized in Table 7. FoR was calculated as a ratio of colonies growing on antibiotic-containing plates to the total number of CFU in the initial test inoculum. The <(inoculum CFU) denotes that no colonies were found at a given MIC (n=3) “Resistant” indicates that a uniform bacterial lawn formed, and colonies could not be counted individually.

The effect of treatment of bacteria with CMB001 was further studied using scanning electron microscopy (SEM), which revealed significant morphological changes following treatment (FIG. 7). For S. aureus, numerous bleb-like structures and debris on the cell surface were visible after cells were treated with CMB001 for 60 minutes. For A. baumanii, dents were apparent after 10 minutes, and undulating deformations and folds were observed after 60 minutes.

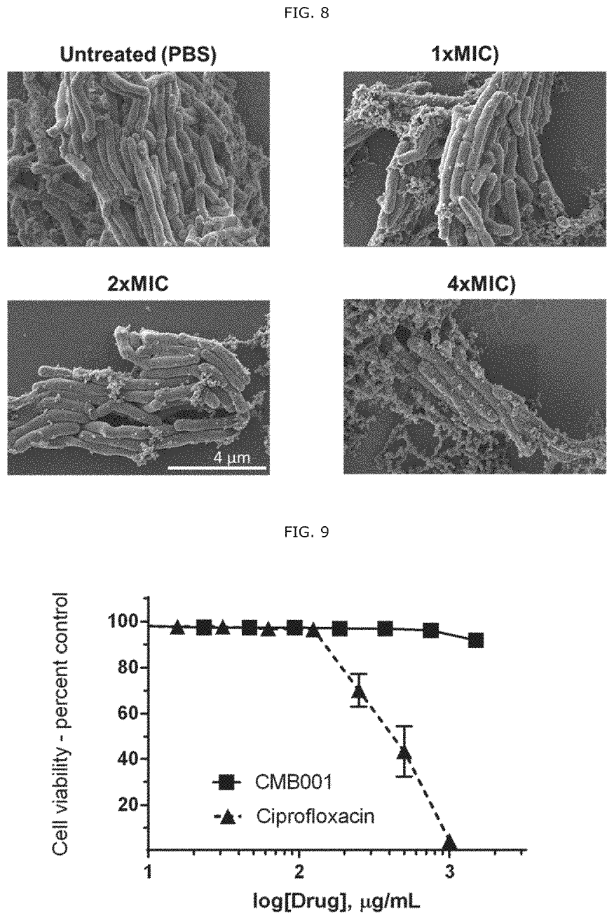

In a similar experiment, M. smegmatis bacteria treated with increasing amounts of CMB001 became wrinkled and covered with irregular debris (FIG. 8).

The exact mechanism of the antimicrobial activity of CMB001 is currently under investigation. Based on morphological changes within the membrane, revealed by SEM images, and considering that CMB001 belongs to lantibiotics, CMB001 may interact with lipid II and/or disrupt the membrane through pore formation.

The toxicity to mammalian cells was tested by applying isolated CMB001 at concentrations of up to 1,500 μg/mL to the J744A.1 mouse cell line. J774A mouse BALB/c cells were grown to confluence and treated with CMB001 for 24 hours. The cell viability was then measured and expressed as a percentage of viable cells treated with vehicle only (without CMB001). Unlike ciprofloxacin, CMB001 did not reduce cell viability (FIG. 9). Similar results were obtained with two other cell lines, Vero and Hep-2, when treated with 200 μg/mL of CMB001, suggesting low toxicity of CMB001 to mammalian cells.

### Example 3. Stability Studies

The thermal and chemical short-term stability of CMB001 was assessed.

For heat treatments, CMB001 (diluted in distilled water to 1 mg/mL) was incubated for 18 hours at 4-60° C.

For pH treatment, CMB001 (5 mg/mL) was incubated for 2 hours at 37° C. at indicated pH levels. Samples were clarified by centrifugation (16,000×g for 5 min) and soluble fractions were tested for antibacterial activity (against S. aureus) and examined by analytical HPLC.

CMB001 retained its full antibacterial activity (MIC) and chemical integrity (Retention Time) after incubation at 4 to 60° C. for 18 hours, and at 37° C. over a wide range of pH (3.0-9.0) (Table 8).

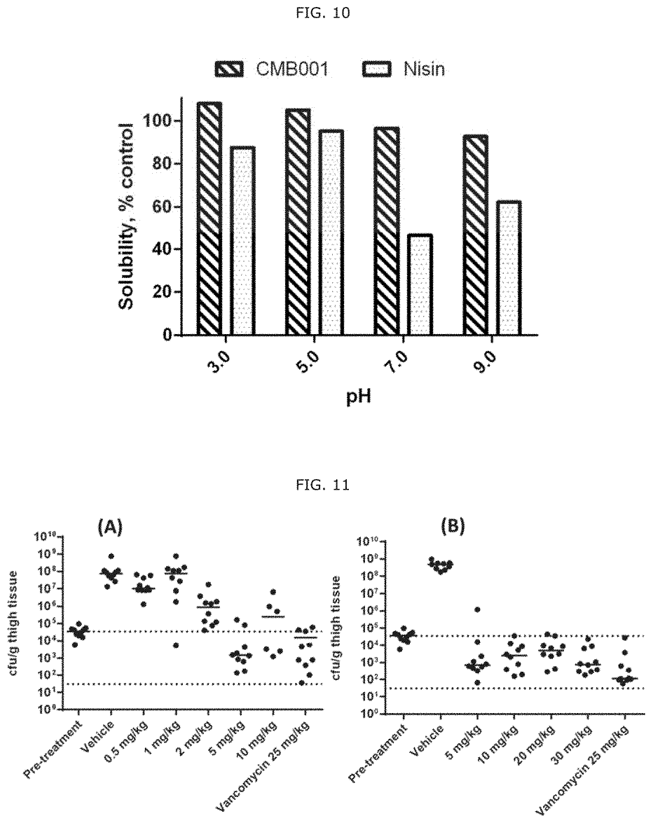

In a separate experiment, 1 mg/mL solutions of CMB001 and nisin were exposed to pH 3-9 for 10 minutes followed by centrifugation. The concentration of peptides in clarified supernatants was measured and the percent solubility calculated relative to the initial concentration. At pH 7.0, the solubility CMB001 was >96% whereas the solubility of nisin was ˜47% (FIG. 10).

Stability studies were also performed in the presence of plasma or serum, or in whole blood. CMB001 retained full antibacterial activity after an 8-hour incubation at 37° C. in human serum or plasma and remained somewhat less active after a 24-hour incubation. Similar results were obtained in mouse plasma (Table 9). It is of particular interest that the MIC of CMB001 in the presence of plasma is ˜10-fold lower than that in water.

In a separate experiment, the stability of CMB001 in plasma was compared to that of nisin. The MIC of CMB001 incubated in 100% plasma or 10% plasma remained 6-8-fold below control values (measured in water). In case of nisin, the MIC measured in plasma at T=0 was only 2-fold lower than that in water and gradually increased throughout the course of the experiment to far exceed that in water. (Table 10)

To assess the stability of CMB001 in whole blood, mouse blood was treated with 400 μg/mL of CMB001, and samples were collected 0-4 hours later. The antimicrobial activity remaining in blood was measured by the diffusion method and expressed as the zone of inhibition (ZOI), proportional to the amount of activity remaining in blood (Table 11). Zone of inhibition (ZOI) is a zone of bacteria free agar plate after depositing 2 μL of a tested sample. In parallel, CMB001 treated blood was centrifuged to remove blood cells and the level of CMB001 was tested in the supernatant (plasma). The antimicrobial activity recovered in plasma was comparable to activity measured in whole blood, and to activity in a sample of plasma spiked with CMB001. This result indicated that CMB001 retains full activity in whole blood and is not adsorbed onto blood cells.

### Example 4. In Vivo Pharmacokinetics (PK)

A single dose PK study in mice provided a baseline pharmacokinetic evaluation of CMB001. The drug concentration in blood samples collected from the caudal vain was quantified by a triple-quad mass spectrometry (MS) with the lower limit of quantification (LLOQ) at 500 ng/mL. Following intravenous (IV) administration of 30 mg/kg, CMB001 remained at detectable levels for at least 60 minutes and the calculated half-life was 0.54±0.15 hours (Table 12). The summary of calculated PK parameters is provided in Table 13.

### Example 5. In Vivo Efficacy

The efficacy of CMB001 was tested in a murine model of thigh infection against methicillin-resistant S. aureus. Mice were rendered neutropenic with two intraperitoneal (IP) injections of cyclophosphamide, 150 mg/kg 4 days before infection and 100 mg/kg 1 day before infection. The immunosuppression regime led to neutropenia starting 24 hours post-administration of the first injection and continued throughout the study. For the efficacy study CMB001 was prepared from frozen stocks of inoculum by dilution in sterile PBS to the desired concentration. Mice were infected with 0.05 mL of inoculum suspension containing S. aureus NRS 384 (USA300-0114) by intramuscular (IM) injection under temporary inhaled anaesthesia (2.5% isofluorane for 3-4 minutes) into both thighs. CMB001 was administered IV at 1, 8 and 15-hours post-infection. The animals were euthanized by overdose of pentobarbitone when they reached clinical endpoints (19-25 hours post-infection). The thighs, from the knee to the hip, including the bone were removed and weighed. Thigh samples were homogenized in 3 mL ice cold sterile PBS containing 10% glycerol and 2.8 mm zirconium oxide beads using a Precellys bead beater set to two cycles of 6,000 rpm for 15 seconds with a five second rest period. In the vehicle treated group a robust infection was established, and all mice reached the clinical endpoint.

In the first efficacy experiment, CMB001 was administered between 5 and 30 mg/kg. Most CMB001-treated animals survived until the end of the study at 25 h post-infection and showed mild to moderate signs of infection. Significant reduction of thigh burden was observed as compared to the vehicle-treated group (P<0.0001). At all doses, the burden was reduced below pre-treatment levels. Lack of a dose response indicated that the maximum efficacy had been reached (FIG. 11A).

In the second efficacy experiment, doses of CMB001 ranged from 0.5 to 10 mg/kg. Treatment with CMB001 led to a dose dependent reduction of bacterial burden compared to the vehicle control (FIG. 11B). In both studies, vancomycin administered at 25 mg/kg reduced bacterial load to or below pre-treatment levels.

Taken together, CMB001 is a novel antimicrobial peptide (AMP) with characteristics required for application as an antibiotic. It is active against multiple MDR pathogens, including methicillin-resistant S. aureus (MRSA), vancomycin-resistant E. faecium, and A. baumannii, and against biofilms. The results described above suggest that CMB001 can be scaled up for manufacturing, is stable to environmental conditions as well as in plasma, serum or whole blood, is active against a range of gram-positive and some gram-negative bacteria and has no known toxicity to mammalian cells. CMB001 shows in vivo efficacy and could provide a beneficial treatment option in comparison with conventional antibiotics such as vancomycin.

Although the invention is illustrated and described herein with reference to specific embodiments, the invention is not intended to be limited to the details shown. Rather, various modifications may be made in the details within the scope and range of equivalents of the claims without departing from the invention.

TABLE 1
Structure quality of CMB001 ensemble structure.
Summary of conformationally restricting experimental constraints

| NOE-based distance constraints: | |
|---|---|
| Total | 616 |
| intra-residue [i = j] | 187 |
| sequential [\| i-j \| = 1] | 176 |
| medium range [1 < \| i-j \| < 5] | 213 |
| long range [\| i-j \| ≥5] | 40 |
| NOE constraints per restrained residue | 19.25 |
| Dihedral-angle constraints: | 40 |
| Total structures computed | 15 |
| Number of structures used | 8 |
| Distance violations/structure | |
| 0.1-0.2 Å | 0.75 |
| 0.2-0.5 Å | 1.13 |
| >0.5 Å | 0.5 |
| RMS of distance violation/constraint | 0.044 Å |
| Maximum distance violation 0.71 A | 0.71 Å |
| RMS of dihedral angle restraint/structure | 16.4° |
| Maximum dihedral violation | 33.3° |
| Structure Quality Factors-overall statistics | |
| MolProbity clashscore | 10.8 |
| Ramachandran Plot Statistics from Molprobity | |
| Most favoured regions | 54.9% |
| Allowed regions | 84.8% |
| Disallowed regions | 15.2% |

TABLE 2
Minimum inhibitory concentration (MIC) of CMB001
against clinically relevant bacterial pathogens.

| Bacteria | MIC (μg/mL) |
|---|---|
| Gram Positive | |
| S. aureus | 1.7 |
| E. faecalis | 3.4 |
| Methicillin-resistant S. aureus | 1.6 |
| Vancomycin-resistant E. faecium | 3.1 |
| Bacillus anthracis | 8 |
| Clostridium difficile VPI 10463 | 2 |
| Gram Negative | |
| Multi-drug resistant A. baumannii | 10 |
| Multi-drug resistant Ps. aeruginosa | ~200 |
| Extended-spectrum beta-lactamase | ~200 |
| producing K. pneumonia | |
| Extended-Spectrum Beta Lactamase E. coli | ~50 |
| E. coli DC0 | 50 |
| E. coli DC2 | 50 |
| Yersinia pestis | 16 |
| Mycobacteriaceae | |
| M. tuberculosis (Mtb H37Rv) | 0.3 |

TABLE 3
Minimum inhibitory concentration (MIC) of CMB001 against clinical
strains representative for the Staphylococcus genus (group).

| | | | CMB001 | Methicillin |
|---|---|---|---|---|
| Hospital | Drug | | MIC | MIC |
| ID | Resistance | 16S ID | (μg/mL) | (μg/mL) |
| CH-01 | N | Staphylococcus aureus strain NBRC 100910 | 1.56 | 1 |
| CH-02 | N | Staphylococcus aureus strain NBRC 100910 | 1.56 | 1 |
| CH-03 | N | Staphylococcus aureus strain NBRC 100910 | 3.13 | 1 |
| CH-04 | N | Staphylococcus aureus strain NBRC 100910 | 3.13 | 2 |
| CH-05 | N | Staphylococcus aureus strain NBRC 100910 | 0.78 to 1.56 | 1 |
| CH-06 | N | Staphylococcus aureus strain NBRC 100910 | 1.56 | 1 |
| CH-07 | N | Staphylococcus aureus strain NBRC 100910 | 3.13 | 1 |
| CH-08 | N | Staphylococcus aureus strain NBRC 100910 | 3.13 | 1 |
| CH-09 | N | Staphylococcus aureus strain NBRC 100910 | 1.56 | 8 |
| CH-10 | N | Staphylococcus aureus strain NBRC 100910 | 1.56 | 1 |
| CH-11 | Y | Staphylococcus aureus strain NBRC 100910 | 3.13 | 32 |
| CH-12 | Y | Staphylococcus aureus strain NBRC 100910 | 1.56 | 16 |
| CH-13 | Y | Staphylococcus aureus strain NBRC 100910 | 1.56 to 3.13 | >64* |
| CH-14 | Y | Staphylococcus aureus strain NBRC 100910 | 1.56 | >64* |
| CH-15 | Y | Staphylococcus aureus strain NBRC 100910 | 1.56 | >64* |
| CH-16 | Y | n/a | 1.56 to 3.13 | 64 |
| CH-17 | Y | Staphylococcus aureus strain NBRC 100910 | 1.56 to 3.13 | 16 |
| CH-18 | Y | Staphylococcus aureus strain NBRC 100910 | 3.13 | — |
| CH-19 | Y | Staphylococcus haemolyticus strain JCM 2416 | 6.25 | 8 |
| CH-20 | Y | Staphylococcus aureus strain NBRC 100910 | 3.13 | 4 |

TABLE 4
Minimum inhibitory concentration (MIC) of CMB001 against clinical
strains representative for the Acinetobacter genus (group).

| | | | CMB001 | Imipenem | Colistin |
|---|---|---|---|---|---|
| Hospital | Drug | | MIC | MIC | MIC |
| ID | Resistance | 16S ID | (μg/mL) | (μg/mL) | (μg/mL) |
| CH36 | N | Acinetobacter baumannii | 10 | 250 | 3.91 |
| CH37 | N | Acinetobacter calcoaceticus | 10 | 0.5 | 3.91 |
| CH38 | N | Acinetobacter baumannii | 40 | 125 | 3.91 |
| CH39 | N | Acinetobacter baumannii | 40 | 15.63 | 3.91 |
| CH40 | N | Acinetobacter baumannii | 40 | 31.25 | 5.86 |
| CH41 | N | Acinetobacter ursingii | 10 | 1 | 1.95 |
| CH42 | N | Acinetobacter guillouiae | 20 | 7.81 | 3.91 |
| CH43 | N | Acinetobacter modestus | 5 | 0.5 | 7.81 |
| CH44 | N | Acinetobacter septicus | 5 | 1.46 | 1.95 |
| CH45 | Y | Acinetobacter baumannii | 20 | 125 | 3.91 |
| CH46 | Y | Acinetobacter baumannii | 10 | 250 | 3.91 |
| CH47 | Y | Acinetobacter baumannii | 20 | 3.91 | 3.91 |
| CH48 | Y | Acinetobacter baumannii | 20 | 7.81 | 3.91 |
| CH49 | Y | Acinetobacter baumannii | 40 | 62.5 | 3.91 |
| CH50 | Y | Acinetobacter baumannii | 20 | 62.5 | 5.86 |
| CH51 | Y | Acinetobacter baumannii | 30 | 31.25 | 3.91 |
| CH52 | Y | Acinetobacter baumannii | 20 | 31.25 | 3.91 |
| CH53 | Y | Acinetobacter baumannii | 20 | 31.25 | 3.91 |
| CH54 | Y | Acinetobacter baumannii | 20 | 7.81 | 5.86 |
| CH55 | Y | Acinetobacter baumannii | 10 | 46.88 | 7.81 |

TABLE 5
Minimum inhibitory concentration (MIC) of CMB001 against clinical
strains representative for the Enterococcus genus (group).

| | | | CMB001 |
|---|---|---|---|
| Hospital | Drug | | MIC |
| ID | Resistance | 16S ID | (μg/mL) |
| CH-26 | N | Enterococcus faecalis strain NBRC 100480 | 6.2 |
| CH-27 | N | Enterococcus faecalis strain NBRC 100480 | 12.5 |
| CH-28 | N | Enterococcus faecalis strain NBRC 100480 | 12.5 |
| CH-29 | N | Enterococcus faecalis strain NBRC 100480 | 6.2 |
| CH-30 | N | Enterococcus faecalis strain NBRC 100480 | 6.2 |
| CH-31 | Y | Enterococcus faecalis strain NBRC 100480 | 12.5 |
| CH-32 | Y | Enterococcus faecium strain NBRC 100486 | 6.25 |
| CH-33 | Y | Enterococcus faecium strain NBRC 100486 | 6.25 |
| CH-34 | Y | Enterococcus faecium strain NBRC 100486 | 3.1 |
| CH-35 | Y | Enterococcus faecalis strain NBRC 100480 | 6.25 |

TABLE 6
Minimum inhibitory concentration (MIC) of CMB001
against drug-resistant clinical isolates.

| | Drug | MIC range, | MIC90 | |
|---|---|---|---|---|
| Strain | resistance | (μg/mL) | (μg/mL) | N |
| S. aureus | susceptible | 0.8-3.1 | 3.1 | 10 |
| S. aureus | MRSA | 1.6-6.3 | 3.1 | 10 |
| E. faecalis | susceptible | 6.2-12.5 | 12.5 | 5 |
| E. faecalis | MDR | 3.1-12.5 | 6.3 | 5 |
| A. baumanni | susceptible | 3.4-27.7 | 27.5 | 10 |
| A. baumanni | MDR | 6.9-27.5 | 27.5 | 10 |

TABLE 7
Frequency of resistance (FoR) to CMB001 in S. aureus
(ATCC29213), MRSA and A. baumannii.

| | | Resistance to: | | | |
|---|---|---|---|---|---|
| MIC | Strain | CMB001 | Nisin | Methicillin | Colistin |
| 2X | S. aureus (ATCC #29213) | 1.3 × 10⁻⁸ | resistant | <3.72 × 10⁻¹⁰ | n/a |
| | MDR S. aureus (CH-11) | 3.5 × 10⁻⁹ | resistant | resistant | n/a |
| | A. baumannii (CH-46) | 1.1 × 10⁻⁷ | n/a | n/a | 2.7 × 10⁻⁷ |
| 3X | S. aureus (ATCC #29213) | 2.2 × 10⁻⁹ | resistant | <3.72 × 10⁻¹⁰ | n/a |
| | MDR S. aureus (CH-11) | 1.8 × 10⁻⁹ | resistant | resistant | n/a |
| | A. baumannii (CH-46) | 2.9 × 10⁻⁹ | n/a | n/a | 3.7 × 10⁻⁸ |
| 4X | S. aureus (ATCC #29213) | 2.6 × 10⁻⁹ | resistant | <3.72 × 10⁻¹⁰ | n/a |
| | MDR S. aureus (CH-11) | 2.9 × 10⁻⁸ | resistant | resistant | n/a |
| | A. baumannii (CH-46) | 1.2 × 10⁻¹¹ | n/a | n/a | 5.2 × 10⁻⁸ |
| 5X | S. aureus (ATCC #29213) | 3.7 × 10⁻¹⁰ | resistant | <3.72 × 10⁻¹⁰ | n/a |
| | MDR S. aureus (CH-11) | 5.3 × 10⁻¹⁰ | resistant | resistant | n/a |
| | A. baumannii (CH-46) | <1 × 10⁻¹¹ | n/a | n/a | 5.4 × 10⁻⁸ |
| 10X | S. aureus (ATCC#29213) | <3.7 × 10⁻¹⁰ | 5.7 × 10⁻⁸ | <3.72 × 10⁻¹⁰ | n/a |
| | MDR S. aureus (CH-11) | <3.2 × 10⁻¹⁰ | resistant | resistant | n/a |
| | A. baumannii (CH-46) | <1 × 10⁻¹¹ | n/a | n/a | 2.0 × 10⁻⁹ |

TABLE 8
Thermal and chemical (pH) stability of CMB001.

| Treatment | MIC (μg/pL) | Retention Time |
|---|---|---|
| 4° C. | 1.6 | 12.1 |
| 22° C. | 1.6 | 12.1 |
| 37° C. | 0.8 | 12.1 |
| 60° C. | 1.6 | 12.1 |
| pH 3 | 0.8 | 12.2 |
| pH 5 | 0.8 | 12.2 |
| pH 7 | 0.8 | 12.1 |
| pH 9 | 0.8 | 12.1 |

TABLE 9
Stability of CMB001 in the presence of serum or plasma.

| | Human | Human | Canine | Mouse | |
|---|---|---|---|---|---|
| Incubation | Plasma | Serum | plasma | plasma | dH₂O |
| time | MIC | MIC | MIC | MIC | MIC |
| hours | (μg/pL) | (μg/pL) | (μg/pL) | (μg/pL) | (μg/pL) |
| 0 | 0.3 | 0.5 | 0.5 | 0.5 | 4 |
| 2 | 0.3 | 0.4 | 0.8 | 0.5 | n/d |
| 4 | 0.3 | 0.8 | 1.0 | 0.5 | n/d |
| 6 | 0.4 | 0.8 | 1.0 | 0.5 | n/d |
| 8 | 0.3 | 0.6 | 1.0 | 0.5 | 2 |
| 16 | 0.3 | 0.9 | 1.0 | 0.5 | 4 |
| 24 | 0.3 | 1.9 | 1.0 | 1.0 | 2 |

n/d not determined.

TABLE 10
Comparative analysis of plasma stability of CMB001 and nisin.

| | CMB001, MIC [μg/mL] | | | Nisin, MIC [μg/mL] | | |
|---|---|---|---|---|---|---|
| Time, | 100% | 10% | | 100% | 10% | |
| hours | plasma | plasma | H20 | plasma | plasma | H2O |
| 0 | 0.25 | 0.25 | 2 | 2 | 3 | 4-8 |
| 4 | 0.38 | 0.38 | 2 | 4 | 5.5 | 4-8 |
| 8 | 0.5 | 0.38 | 2-3 | 12 | 13 | 4-8 |
| 16 | 0.6 | 0.5 | 2-3 | 14 | 24 | 4-8 |
| 24 | 0.5 | 0.7 | 2-3 | 32 | 22 | 4-8 |

TABLE 11
Stability of CMB001 in whole blood and plasma.

| Incubation | Whole | Plasma | Spiked |
|---|---|---|---|
| time | blood | fraction | plasma control |
| minutes | ZOI, mm | ZOI, mm | ZOI, mm |
| 10 | 10 | 11 | 9 |
| 60 | 9 | 9 | 11 |
| 120 | 8 | 8 | 11 |
| 180 | 8 | 8 | 11 |
| 240 | 8 | 7 | 10 |

400 μg/mL CMB001 added to whole blood

TABLE 12
Summary of CMB001 in blood following IV
dosing to male CD1 mice at 30 mg/kg.

| Nominal | | Standard | | Standard |
|---|---|---|---|---|
| Sampling | Mean | deviation | Mean | deviation |
| Timepoint | Concentration | (SD) | Concentration | (SD) |
| (hours) | (ng/mL) | (ng/mL) | (nM) | (nM) |
| 0.08 | 4766 | 1423 | 1424 | 425 |
| 0.25 | 2453 | 945 | 733 | 282 |
| 0.50 | 1281 | 335 | 383 | 100 |
| 1.00 | 916 | 76 | 274 | 23 |
| 2.00 | BLQ* | — | — | — |
| 4.00 | BLQ | — | — | — |
| 8.00 | BLQ | — | — | — |
| 24.00 | BLQ | — | — | — |

*BLQ: below the limit of quantification.

TABLE 13
Single dose PK (30 mg/kg) summary of PK parameters

| | CMB001 IV 30 mg/kg Blood | | | | |
|---|---|---|---|---|---|
| PK Parameter | 1 | 2 | 3 | Mean/Median | SD |
| Dose (mg/kg) | 30.00 | 30.00 | 30.00 | 30.00 | 0.00 |
| C0/Cmax (ng/mL) | 11254 | 4449 | 5207 | 6970 | 3729 |
| C0/Cmax (nM) | 3363 | 1330 | 1556 | 2083 | 1114 |
| Clast (ng/mL) | 834 | 985 | 928 | 916 | 76 |
| tlast (h) | 1.00 | 1.00 | 1.00 | 1.00 | |
| t1/2 (h) | 0.64 | 0.43 | — | 0.54 | 0.15 |
| MRT (h) | 0.71 | 0.63 | — | 0.67 | 0.05 |
| Vdss (L/kg) | 7.0 | 6.6 | — | 6.8 | — |
| CL/CL_F | 166 | 175 | — | 170 | — |
| (mL/min/kg) | | | | | |
| AUCinf (ng · hr/mL) | 3014 | 2856 | — | 2935 | — |
| AUCinf (nM · hr) | 901 | 854 | — | 877 | — |
| AUC0-t (ng · hr/mL) | 2243 | 2242 | 1624 | 2036 | 357 |
| AUC0-t (nM · hr) | 670 | 670 | 485 | 608 | 107 |
| Number of Points | 3 | 3 | — | 3 | — |
| used for Lambda z | | | | | |
| AUC % | 26 | 22 | 41 | 29 | 10.4 |
| Extrapolation to | | | | | |
| infinity | | | | | |
| AUC % Back | 23 | 12 | 13 | 16 | 6.0 |
| Extrapolation to C0 | | | | | |

Dose (mg/kg): Amount of drug administered
C0: initial plasma drug concentration at time zero following injection
Cmax: Maximum (peak) plasma drug concentration
Clast: Last measurable plasma concentration
Tlast: Timepoint of last measurable plasma concentration
t1/2: Elimination half-life
MRT: Mean residence time
Vdss: volume of distribution at steady state
CL: The volume of plasma cleared of the drug per unit time
AUCinf: Area under the plasma concentration-time curve from time zero to infinity
AUC0-t: Area under the plasma concentration-time curve from time zero to time t

1. A method of inhibiting growth of or killing microbial cells, comprising administering to the microbial cells an effective amount of a composition comprising a peptide, wherein the peptide consists of an amino acid sequence selected from the group consisting of SEQ ID NOs: 1 and 4-19 or an amino acid sequence at least 90% homologous to an amino acid sequence selected from the group consisting of SEQ ID NOs: 1 and 4-19.

2. (canceled)

3. A method of treating a subject infected by microbial cells, comprising administering to the subject an effective amount of a composition comprising a peptide, wherein the peptide consists of an amino acid sequence selected from the group consisting of SEQ ID NOs: 1 and 4-19 or an amino acid sequence at least 90% homologous to an amino acid sequence selected from the group consisting of SEQ ID NOs: 1 and 4-19.

4. The method of claim 1, wherein the peptide consists of SEQ ID NO: 1.

5. The method of claim 1, wherein the microbial cells are selected from the group consisting of Staphylococcaceae, Streptococcaceae, Enterococcaceae, Moraxellaceae, Peptostreptococcaceae, Mycobacteriaceae, Pseudomonadaceae, Enterobacteriaceae, Bacillaceae, Yersiniaceae, fungi and combinations thereof.

6-10. (canceled)

11. The method of claim 1, wherein the composition further comprises an additional antimicrobial agent.

12. (canceled)

13. The method of claim 1, wherein the composition further comprises a potentiator.

14. (canceled)

15. The method of claim 1, wherein the composition further comprises a stabilizer.

16-19. (canceled)

20. The method of claim 1, wherein the microbial cells are in a biofilm, further comprising administering the composition into the biofilm.

21. The method of claim 1, wherein the microbial cells are on a surface, further comprising administering the composition to the surface.

22. The method of claim 21, wherein the surface is on a medical device or medical equipment.

23. (canceled)

24. An isolated peptide consisting of an amino acid sequence selected from the group consisting of SEQ ID NOs: 1 and 4-19 or an amino acid sequence at least 90% homologous to an amino acid sequence selected from the group consisting of SEQ ID NOs: 1 and 4-19.

25. The isolated peptide of claim 24, wherein the peptide consists of the amino acid sequence of SEQ ID NO: 1.

26. A composition comprising the peptide of claim 24 in an antimicrobial effective amount for inhibiting growth of or killing microbial cells.

27-34. (canceled)

35. The composition of claim 26, further comprising an additional antimicrobial agent.

36. (canceled)

37. The composition of claim 26, wherein the composition further comprises a potentiator.

38. (canceled)

39. The composition of claim 26, wherein the composition further comprises a stabilizer.

40. (canceled)

41. (canceled)

42. The composition of claim 26, wherein the microbial cells are in or on a subject in need thereof.

43. (canceled)

44. (canceled)

45. The composition of claim 26, wherein the microbial cells are in a biofilm.

46. The composition of claim 26, wherein the microbial cells are on a surface.

47. The composition of claim 46, wherein the surface is on a medical device or medical equipment.

48-53. (canceled)
